(19) United States
(12) Patent Application Publication
(10) Pub. No.: US 2020/0168326 A1
(43) Pub. Date: May 28, 2020

(54) APPARATUS AND METHOD FOR LINKING AND EVALUATING INFORMATION IN CLINICAL DATA IN A MEDICAL DEVICE

(71) Applicant: W.O.M. WORLD OF MEDICINE GMBH, Berlin (DE)

(72) Inventors: Peter WESTERHOFF, Berlin (DE); Julia GRUNDMANN, Berlin (DE); Yves KOETH, Berlin (DE); Matthias HILDEBRAND, Berlin (DE); Hans-Joachim CAPPUIS, Berlin (DE)

(21) Appl. No.: 16/632,904

(22) PCT Filed: Jul. 10, 2018
(86) PCT No.: PCT/DE2018/000210

§ 371 Date: Jan. 22, 2020

(30) Foreign Application Priority Data
Jul. 28, 2017 (DE) 10 2017 007 100.4
Dec. 29, 2017 (DE) 10 2017 012 063.3

(51) Int. Cl. G16H 40/63; A61B 90/90; A61M 13/00

(52) U.S. Cl. CPC G16H 40/63; A61M 2205/52; A61M 13/003; A61B 90/90

(57) ABSTRACT

The subject matter of the invention is a medical engineering device for use in minimally invasive surgery (MIS). Until now, minimally invasive surgery (MIS) as a device solution is isolated from the networked components of the operating room. The medical professionals are to be assisted in the device setting by an assistance system, by a better integration of the devices used for the MIS (e.g., insufflators, pumps, cameras, monitors) in the total surgery process as well as by a manufacturer-independent, open approach for information collection, information evaluation, and information-based process optimization. In the pre-operative phase already, before the actual surgical intervention, a multitude of information can be collected or acquired, in order to make them available in the intra-operative phase. At the same time, on this basis of data, a first individualization of the diagnostics or therapy to the respective patient can be performed.

### SUBJECT MATTER OF THE INVENTION

Field of application is the use of data from networked devices in minimally invasive surgery, patient data and/or previous experiences from databases to assist the surgeons in the setting of the devices as well as in the ongoing control of the devices by means of the data.

The solution according to the invention relates to concepts for a device-internal use of comprehensive networking in the clinical environment of surgical interventions for diagnosis or therapy.

Herein, “networking” means the comprehensive use of information of all relevant data present in the operating room. A “medical device” is every energy-operated or non-energy-operated technical device that is used for diagnostic or therapeutic interaction with the human or animal body.

### PRIOR ART

Prior art relating to the field of minimally invasive surgery (MIS) currently comprises a device system of several individual components. The access for medical devices to the surgery site of the patient is achieved by a trocar-optics combination. The trocar represents the seal of the body's exterior with a defined access for instruments and can tightly be closed by valves. The trocar is connected via a hose system with a pumping device. With this pumping device or fluid pump, during surgical interventions in body joints or other body cavities (for example, bladder, rectum, uterus, joints, abdomen, or other artificially created or natural openings in the human body), a rinsing medium (liquid or gaseous) is pumped via the hose connection for expansion into the surgery site.

When a liquid is used as the rinsing medium, a surgical instrument such as a shaver is introduced via another access into the patient. Often, a drain is connected, which removes the rinsing liquid together with blood and tissue residues from the patient.

For another intervention in the field of MIS, the expansion of the abdominal cavity with a gaseous medium is required. Here, too, a trocar is used for the access of the instruments, of which at least one is used for the supply of the gaseous medium and often another one for the discharge of the gaseous medium. These two functionalities of supply and discharge may also be combined to a single device. The supply and discharge also occur via hose systems and are controlled by a pumping device or an insufflator.

The object of the device-internal control of the two aforementioned types of pumping devices is to build up and maintain a defined pressure at the surgery site, while a desired volume flow can be set. For controlling the pumping devices, a few purely technical measured quantities are available. A current challenge is, therefore, to adjust the pressure at the surgery site in an optimum manner to the individual patient and also to maintain it in the case of disturbances.

The control of pumps and insufflators has already achieved a high technical level, which is hard to improve without the involvement of further data and influencing parameters via a networked interface.

Generally, an obvious solution for the use of data of the treated patient is a manual entry in the control panel of the medical device. This is prone to faults and is time-consuming. The processes in the operating room do not make such an entry into the medical devices beforehand appear time-efficient, since patients are also treated on short notice in other similarly equipped operating rooms.

Any time savings can directly be converted into cost savings, thereby the hospital and finally, also the health system is financially disburdened.

By the implementation of additional data interfaces according to the invention, medical devices will gain functionality, usability, performance, but also safety.

The approaches existing as prior art in the field of medical networking relate to the communication structures for networking of devices, however, not to the use of the data by the medical devices. The activities compiled concerning the networking under OR.Net deal with the development of open interface standards for communication of medical devices among each other. Similar approaches have been launched in the USA under the term MD PnP. Within the operating room, various devices still operate autonomously today, which is mainly due to non-uniform interfaces of the different manufacturers of medical products. These open interfaces represent an extensibility that can be achieved by underlying definitions in a manufacturer-independent and device-open manner. Different are the closed proprietary communication solutions of manufacturers such as Stryker: SIDNE—Richard Wolf: Core Nova—Storz: OR-1—Dräger: Infinity—Olympus: Endoalpha—Maquet: Tegris—and others who do not lay all communication protocols and provided data open.

WO 2009/032 134 describes a method and a device to transport data of an implanted medical device via a wireless connection to a network to which a server, surgeon stations, and programming stations are connected. This reference is composed of several inventions, of which the method is disclosed in claims 38-41, wherein a mutual authentication of a portable patient communication device and a remote server and a data exchange following after successful authentication is performed. The implementation of such functionality is not part of the solution according to the invention and can be enabled, without limiting the solution according to the invention, in a different way according to prior art.

In WO 2014/040175 is described a server-based bio-signal transmission (of unspecific origin) with feature extraction and self-learning or supervised learning functions, as well as a method and an internet-connected network with server-based services. These are limited, in this specification, to include at least data from a brain-computer interface. In the solution according to the invention, a use of a server is not provided, but just to the contrary the networking among each other, without the function of a superordinate control computer, referred to as a server, for exchange and data management.

Disadvantages are also caused by that there are for medical devices, mainly in the field of the imaging methods, interfaces defined as open, by means of which patient data and image data can be transmitted, namely HL7 and DICOM. These are technically not intended to enable operation of devices or to exchange information of devices in an operating room. Items such as time-critical data provision or even real-time capability as well as alarm management are not implemented.

What concerns information technology, device systems were heretofore isolated from each other in the operative environment. Networking of all components offers enormous advantages for the improvement of patient therapy, since
more data are available,
by redundant data, the safety against sensor failures is increased,
sensors can be saved,
device settings tailored to the patient's individual needs can be employed.

For this networking, the necessary interfaces and communication protocols must be present in the medical devices.

In the field of MIS, all devices used in the surgical process are currently manually detected and documented in the pre-operative phase. So far, the device configuration and the set value adjustment are performed purely manually by clinical personnel based on personal experiences or manufacturer instructions.

So far, the settings are performed within the operating room and during the medical procedure directly at the device. The device operation is, however, difficult to perform due to the position close to the operating table and at the same time in the non-sterile area of the operating room. Persons, who were physically able to operate the device, can only poorly reach the operating elements, since they are nearly in the sterile area of the operating room. Sterile personnel, on the other hand, only poorly reach the operating elements of the devices, the medical devices in most cases not being sterile and the sterility in the sterile area being ensured by sterile coatings. Therefore, the correct setting is currently frequently neglected, and standard parameters are used. An instruction for device operation of the sterile surgeon to the surgical nurse who is in the unsterile area is obvious. This will disturb courses of the medical procedures by technical processes, and the success of a change of the settings strongly depends on the knowledge and the understanding of the surgeon/nurse team.

Anamnesis data are collected and recorded before every surgery. Up to now, the use of these a priori data has only been carried out manually by the clinical personnel, for example, by changing the device parameters for older or younger patients in a targeted manner. An automated evaluation by classification methods, in order to better tailor the therapy to the individual patient, is currently not used.

Focusing the problem on the MIS, a surgical technique that in contrast to conventional methods achieves access to the patient via smallest wound incisions and offers many advantages for the patient by the little lesions of the skin layers and reduces complications such as traumata, scarring, or infections and achieves shorter periods of recovery, with poor regulation of the internal pressure in body and joints, disadvantages by intra-operatively occurring swellings, post- and intra-operative complications as well as insufficient vision for the surgeon during an intervention will be caused.

Currently, the use of pre-information about the patient or current information on the condition of the patient for carrying-out the MIS is made exclusively by the medical professionals in the form of subjective adaptations of the device parameters or settings. An automatic data exchange between the MIS and other components in the operating room does not take place. So a large number of technical settings based on empirical values must be made by the surgeon.

WO 2015/069,182 A1 describes a supplement of the control device of a pumping device by a blood pressure measuring device in an apparatus for the purpose of improved control of the pressure in a body cavity (pressure as low as possible, but high enough to prevent blood leakage from injured blood vessels). The solution described in this specification describes in a specific embodiment the use of data of a networked and separate apparatus, e.g., of a vital monitor, which can use data such as blood pressure in an apparatus with pumping device, such as a roller pump, for a similar purpose.

Furthermore, WO 2015/069,182 describes a method, wherein the measured blood pressure value is multiplied by a correlation factor, in order to determine at the surgery site the blood pressure of the vessels (true perfusion pressure), thus controlling the supply pressure of the pumping device.

The difference to the solution according to the invention described in this specification thus resides in a communication layer between two independent medical devices not necessarily coming from the same manufacturer that exchange data to effect such a control corresponding to the teaching of WO 2015/069,182. The solution according to the invention described in this specification also includes the possibility of the functionality WITHOUT such a connection or with a connection being separated in the course of the medical procedure.

In an attempt to avoid these disadvantages by changing the settings of the device by the surgeon or the surgery nurse, an increased communication effort, distraction, high workload is caused, and ultimately a concentration disorder of the surgeon will be the consequence.

The better networking, the inclusion of different data sources and sensors offer advantages for minimally invasive surgery over prior art with conservative control technique and conventional, not automated information management.

These advantages are
improvement of regulation and control of medical devices,
adaptation of the therapy to the patient,
automated recording and evaluation of patient and surgery data,
implementation of an assistance system for the medical personnel,
fault detection for devices and patient monitoring.

During the actual surgical intervention, the patient therapy is improved by a patient-specific control of relevant physiological state quantities compared to the previous state. At the same time, the risk of complications is minimized and the intervention time is reduced. The surgery time is shortened by automatic pre-settings that otherwise could be done manually. These are based on that expert knowledge, patient information, and surgery data are combined.

During a clinical procedure, several medical devices are acting directly or indirectly at the same surgery site, and influence each other. This is most evident in the field of the interventions in body cavities, which are expanded by a medium and are thus under pressure. Medical devices for the expansion are pumps or insufflators. Medical devices, whose object is to remove material, suck this pressurized medium off, or generate a leakage path, on which the medium can escape. Thus, the fluid resistances for the introduction and the escape or suction of the medium are quantities that influence a control, but are not always clearly coupled to each other in the current medical routine of sensor data detected with a medical device.

At present, in an artificial situation before introduction of the medical devices into the body cavities, such fluid resistances are measured from pressure and volume of a fluid exiting into the free environment, i.e., the static pressure/flow characteristic, and therefrom an assumption about the existing fluid resistance to the body cavity is determined. With this assumption, the situation in the body cavity is calculated using the technical parameters detected during the medical procedure at the pumping device and is displayed to the surgeon.

The disadvantage of this method is that it is relatively time-consuming and the surgeon cannot compensate this period of time with a benefit and, therefore, will often not perform such a pre-recognition.

An aid for the device configuration or a warning display that can issue context-sensitive recommendations for action, is only very limitedly possible in medical devices. A device configuration can only be made by an additional selection of usage scenarios, such as the body region and the type of procedure, for example, by a selection in the device that is used for a knee surgery and therapeutic intervention. Systems that use sensors and thus recognize a context, are known from other areas, which cannot be employed, however, in medicine because of the complex contexts. The use of vital parameters detected outside of the device for the generation of warnings or recommendations for action is not known.

For a self-organizing networking, relevant examples are known that, however, have not been used up to now in the field of medical engineering.

Further prior art can be found in the following documents:

EP 2662106 B1 describes an insufflation device, which comprises a dissection mode as well as an insufflation mode. The insufflation device includes an insufflator and a measurement, control and regulation device for controlled and regulated discharge of the gas. The insufflator is fluidically connected via a gas supply line to a pneumatic tissue dissection probe. The tissue dissection probe is electronically connected to the measurement, control and regulation device. There is provided a switching device that is configured to provide switching of the regulated and controlled gas flow to the gas output of the tissue dissection probe or of an insufflation cannula. An independent claim is included for a method for an alternating insufflation of gas into a body cavity and a pneumatic dissection at the same time with an insufflation device.

WO 2015/161,965 describes the control of a medical device by means of different modes of operation based on sensor data. The invention relates to a mobile medical device (1) with a portable operating unit that includes a housing (10) with several differently oriented or positioned operating elements at a user interface as well as at least parts of a control device (5). In order to provide the user with substantial assistance, the operating unit comprises a position sensor device (3) that detects the orientation (x, y, z) of the housing (10) with respect to a reference value and correspondingly generates sensor data. With the orientation and different modes of operation, the sensor data can selectively be activated by the control device (5).

WO 2007/065,237 describes an insufflator operation that automatically switches into a safety mode, when there is no regular monitoring signal (page 5 lines 1-3).

DE 19904090 describes a method and a device for automatic control and management (also for documentation purposes) of program-controlled medical devices. The focus of the patent is on the networking and communication of devices with a bus system (CANopen). Herein, the medical devices act as slaves and the main computer acts as a master that can control all medical devices. Different from the solution according to the invention described here, the devices cannot control each other or exchange data. As functions are described a macrorecorder to define a cooperation of devices, a maintenance and service analysis at system start, a log-in-file for certain documentation functions, a fault protocol and treatment. Also described is a construction in a double-ring structure, a ringstar structure or a ring-ring structure.

EP 1995679 describes a device and a method for networking and central operation as well as data transmission for documentation purposes and/or control or parameter adjustment of at least one device that is used during a medical intervention. There is a central database that is kept on a control unit, an external server accessible via networks or a readable-only data carrier.

Paragraph [0016] of EP 1995679 describes that device parameters or device actions are initiated by a signal automatically generated by a sensor. There is, however, not mentioned any technical teaching, except that a device pre-setting is automatically adopted—it is not mentioned based on which sensors this occurs and what they effect. In particular, a regulation of device parameters by sensor values, as in the solution according to the invention presented here, is not described and not obvious.

Each transfer of the database contents into a device is, in the EP 1995679, mandatorily linked with an approval of the surgeon, i.e., the actuation of a confirmation element.

Paragraph [0028] of EP 1995679 describes that data of the patient (age, height, weight, current or already detected diseases, anatomic peculiarities) require altered parameter values, and that device configurations and optimum parameters depend on the employed devices and manufacturers. In paragraph [0029], the provision thereof in the database is described, and different patient-dependent data sets or a formula for the calculation of patient-dependent parameters are described. As a technical teaching, as an example is mentioned the inter- or extrapolation. The solution according to the invention provides an evidence-based approach, i.e. this is not described therein and is not obvious.

EP 2763064 A2 describes an insulin pump with a configurator for setting operating parameters of the insulin pump.

### Solution According to the Invention

In order to deal with the already presented challenges and current deficits, the solution according to the invention provides the use of medical and technical measurement values, which are already available by other device systems in the operating room.

The invention teaches a medical device for use in minimally invasive surgery (MIS), including
at least one interface to at least one other medical device, wherein the interface transmits measurement data and/or patient data,
at least one storage unit that stores the operating parameters obtained via the interface based on measurement data and/or patient data and that further permanently contains at least one data set of operating parameters as a fall-back level,
at least one computation unit connected via the interface in the other medical device, which based on the measurement data and/or the patient data computes the operating parameters of the medical device,

wherein the calculation of the operating parameters takes place under consideration of a priori data selected according to a predetermined criterion, wherein the regulation of the operating parameters of the medical device takes place using the calculated operating parameters.

The object that is solved by the solution according to the invention is a networking of medical devices among each other without a central control device using protocols corresponding to prior art that is used for an improvement of the device performance in the meaning of a better control or regulation, identification of the disburdening of the surgery personnel as well as assistance when selecting the device parameters, the accessories, and a simplified documentation.

The solution according to the invention can also be interpreted as a provision of a medical device (fluid pump), which depending on the situation guarantees an ideal surgery assistance, wherein
the ideal surgery assistance consists of the combination of optimum modes of operation and the respectively related disposables,
the mode of operation is respectively defined by the pressure, the fluid flow, the control parameters, and the setting range,
the selection of the optimum mode is performed in an either patient- or intervention-depending manner,
a recorded matrix is used, in which types of patients and kinds of intervention are listed, weighed and linked with each other, wherein exclusion criteria (not allowed combinations) are observed,
an algorithm that determines or proposes the ideal mode under consideration of the matrix elements,
an adjustment and/or a recognition of employed disposables or other medical devices is performed, and
depending on the result the system is released, an alternative suggestion is calculated, or the system is blocked.

An embodiment of the fluid pump as a pure suction pump or as a pressure-loaded reservoir is also covered by the invention.

The automatic mode selection or a novel gentle mode relate in first place to the following benefits for the user:

a) User-Friendliness

The automatic mode selection disburdens the user of the necessity to perform the settings himself. Further, the risk of operating faults is minimized.

b) Reduction of Pain

The reduced expansion by the insufflation leads to a gentle treatment and thus to an optimum avoidance of pain for the patient.

c) Medical Added Value

The use of modes described here leads to a maximum possible reduction of stress for the patient and may also be the basis for the treatment of risk patients.

The basic idea of the invention can be split into
patient data-reactive device control,
assistance system (disburdening of technical setting parameters, warning of critical conditions, visualization of the process and the remaining margins).

The precondition is the provision of data interfaces to information sources in the operative environment such as the information system of the hospital or the anesthesia system as well as the specification of relevant information sources and of the interfaces.

The solution according to the invention includes
a data communication layer for networking the devices in the surgery,
a data classification of a priori anamnesis data,
an identification method for automatic recognition of employed devices in the pre-operative phase,
a set of characteristic curves for controller presetting based on a priori knowledge,
set value recommendations based on a priori knowledge,
data transmission from the pre-operative phase in the assistance system,
further, for an optimum insufflation, a comparison should be performed, if the disposable chosen in the specific individual case harmonizes with the calculated mode,
provision of a gentle mode, so that it is possible for the surgeon for particularly sensitive cases (infants/seniors), if necessary, to optimize the performance of the device with regard to the patient's well-being.

Data classes (e.g., type of patient, type of surgery) are created, and the respective anamnesis data are analyzed. Set value recommendations derived from the data analysis and controller pre-settings of the medical devices improve the patient therapy and are used in an assistance system for assisting the clinical personnel. The patient therapy is, for example, significantly improved by that bleedings are reduced and/or undesired tissue damages by too high pressures at the surgery site are prevented. The assistance system supports the surgeon by individually adapted recommendations based on patient data and technical measurement values.

According to the invention, origin and regulation parameters of insufflators and pumps in minimally invasive surgery are directly or indirectly changed in the medical device by data input, thereby an improved device performance being guaranteed, which improves the vision at the surgery site, reduces bleedings, and minimizes the burdening of the circulation of the patient.

By the linkage of all pre-, intra-operative patient and surgery information proposed as a solution according to the invention, the surgeon or the clinical personnel can be disburdened of technical configuration procedures of the used devices.

For the solution of the defined problems, according to the invention, the analysis and acquisition of new data takes place in the pre-operative phase as well as the derivation of device configurations from the data.

While the actual surgical intervention is performed, devices of the MIS are up no now isolated and are only linked with clinical information through the adaptations by the operating surgeon. By that all available medical and technical data from different data sources are automatically considered, adaptations of the device parameters and settings should be made, and a better regulation and pre-setting of surgery-relevant parameters should be implemented. All a priori data from the pre-operative phase should be used for an automatic device configuration. In doing so, the device configuration in the form of controller parameters and the set value input should take place automatically.

These include:
parameter regulation when employing non-invasive measurement methods,
inclusion of device activities that affect the function of the medical devices,
suggestion of medical devices supporting the surgery in an optimum manner, or disposables (e.g., hose set),
pre-selection of a mode of operation supporting the surgery in an optimum manner,
adaptation of the regulation parameters to the patient,
use of a monitoring layer and fall-back level,
methods for automatic data processing and classification,
methods for automated device recognition,
methods for a priori controller setting and set value recommendation,
methods for inclusion and use of information sources existing in the operating room, but not included until now.

In order to solve the problems of the inter- and intra-patient variability in the intra-operative phase, a continuous adaptation of the regulations to the patient under consideration of current vital parameters is performed.

According to the invention, an automated anamnesis evaluation is provided that links the current patient data with previous knowledge. The device systems used during the intervention are to be automatically detected by means of identification methods. Furthermore, recommendations for action for the surgeon are generated from all available a priori data.

According to the invention, there is also provided a self-adjustment of the used devices, which disburdens the medical personnel of technical processes and optimizes the therapy tailored to the patient's individual needs. All collected information, set value recommendations and settings are transferred to the intra-operative phase.

Until now, the algorithm-based information analysis before a surgery attracted little attention. In this pre-operative phase, a multitude of patient and surgery-related data are already generated and these can be analyzed. In detail, the following is possible in the pre-operative phase:
acquisition of information by a data interface to the patient management system,
carrying-out an anamnesis evaluation,
carrying-out an automatic device recognition,
controller pre-setting for the surgical intervention,
pre-selection of a mode of operation supporting the surgery in an optimum manner,
suggestion of medical devices supporting the surgery in an optimum manner, or of disposables (e.g., hose set),
issue of set value recommendations for the surgical intervention, and
simple transfer of recommendations and controller pre-settings.

The extended exchange of information between the device systems in the clinical-operative environment is directly used for an optimization of the control technique of the individual medical devices.

The automated information processing analyzes and classifies patient and device data and derives device parameters and device behavior therefrom. The classification process corresponds to established methods, and a suitable classificator structure is created for every field of application. Training and validation of the classificator occurs before the implementation in the medical device or also during the medical procedure or following the medical procedure by, e.g., learning systems by means of data from the clinical environment.

In addition to the use of existing information, process information is collected, analyzed, and provided for other networked medical partial systems.

The field of application of the planned expert system may comprise further functional units in the operating room. As the most important area, anesthesia will enormously benefit from a system of experiences continuously updating itself and the couplings to other clinical devices and systems.

By the data from the anamnesis and device recognition, device configurations can automatically be selected. These settings can be optimized to the patient's individual needs for the respective therapy and thus improve already pre-operatively minimally invasive surgery. The same applies to set value inputs such as the pressure at the surgery site.

For an identification of medical devices connected to the fluid system (shaver, hose set, trocar, etc.) currently the relevant device parameters are used by adoption of different operating states (flow rates and supply pressures in fluid pumps), in order to determine the relevant quantities (flow resistance) or compare them with previously measured quantities. This method is based on the cooperation of the surgery personnel and takes a lot of time. According to the invention, the device identifications from the surgery planning system, a labeling of the medical devices with RFID chips (wireless data transmission) or a visual recognition by image processing of connected devices or other methods can be provided, in order to perform the device settings resulting from the use.

In medical engineering, a core element of any product development is the assurance of the patient's safety. According to the invention, critical states of the patient are to be detected and in case of a fault, the system is to be brought into a safe mode of operation. This object is achieved by a fall-back level.

The fall-back level in the medical device ensures that a safe condition is achieved when data inputs are missing or also failing during the procedure. In doing so, certain quantities are kept, if applicable, other quantities are changed to a safe mode.

All information and the pre-operative controller settings and set value recommendations are also available to the assistance system. This assistance system is available to the medical personnel in the pre- and intra-operative phases and disburdens them of technical processes.

Based on, among others, anamnesis data, type of interventions, type of patient and current vital parameters of the patient, recommendations for action are issued to the clinical personnel by an assistance system according to the invention, such as an initial set pressure recommendation or to increase the set pressure, and/or hints to critical situations are given, such as a longer-lasting exposure to high pressure or a high absorption of fluid in the body. Likewise, a special mode of operation or a special hose (e.g., with wetting or heating) that is most adequate for the coming surgery, can be proposed.

### EMBODIMENTS

The provision of a data interface to the patient management system can be achieved with a built-in device or through a middleware and provide and manage the communication between the different data sources in the operating room. Thus, the devices of the MIS are incorporated in the self-organizing communication network. A pre-sorting process based on clinical expertise divides the a priori data into data classes (e.g., type of patient, type of surgery). For this purpose, a compilation of rules for the controller pre-setting is utilized for the pressure regulation. Potential a priori knowledge is, e.g., the type of surgery, anonymized patient characteristics as age, height, weight, etc., or physical properties of the employed devices. Due to the changing properties of the system of device and patient, a controller selection is to be made, in order to employ a specialized instead of generic controller structure, i.e., a control algorithm explicitly tailored for the control task instead of a generally useful control algorithm that is respectively adapted with parameters to the current object.

An embodiment of an automated information processing evaluates data generated during the procedure by an analysis device or analysis method for the patient's vital parameters. The object is, for example, to adjust the pressure at the surgery site exactly to the blood pressure in the surrounding tissue, which would be ideal from a clinical point of view. Ideal means that by a pressure difference of zero neither bleedings in the control area nor undesired fluid entry into the body by the distensions or rinsing medium will occur. At the same time, an as low pressure as possible at the surgery site is desired, in order to lower the tendency to tissue swellings (arthroscopy), increased pressure, in order to reduce bleedings at the surgery site (arthroscopy and generally endoscopy) The optimum set value for the pressure at the surgery site is determined before the surgical intervention from the vital parameters of the patient. Herein, for example, the age of the patient is an influence quantity, in order to determine from general biomechanical circumstances, such as the pressure resistance of the joint capsule, i.e., the filling pressure before a crack occurs, a set value for the pressure at the surgery site.

During the surgical intervention, too, vital parameters of the patient are used, for example, from the diastolic blood pressure or another value linked therewith, in order to determine set values for the pressure at the surgery site, for example.

In order to achieve this balance between the required boundary conditions such as vision on the joint interior and rinsing and acute or insidious damage by too high pressure, the core element of the control loop, i.e., the method for adaptation to the individual patient, is the inclusion of context information that is made available by the networked data. In a certain surgery context with the regulation of the pressure by means of a dual roller pump, the boundary condition is the maintenance of a volume flow required by the surgeon at the surgery site. Both are achieved, for example, with the approach to include current vital parameters of the patient provided via the network in the control concept of the medical device.

One aspect is the set value adjustment for the pressure at the surgery site. Vital parameters such as the intra-operatively measured blood pressure value, the body temperature, the heart rate or data from the anesthesia are to be taken into account. Furthermore, the set value recommendation is adjusted, also during the surgery, continuously to the individual condition.

Another aspect is the consideration of vital parameters for the control quantity observation or estimation. Current concepts are based on purely technical measurement quantities. With the vital data, biological signals within the control concept are used. This means that from changes of the vital parameters, a control quantity estimation is influenced, thereby additional information being included as a control quantity. For this purpose, in addition to methods of parameter estimation, also the multivariate regression analysis is used. In addition to vital parameters, information also originates from the networking of medical devices among each other and the combination thereof with existing data sources that are common in the hospital. The inclusion of additional data sources can be used in a universal manner for a multitude of control structures.

The provisions of the pre-operatively performed controller pre-setting should be checked and, if necessary, be adjusted, in order to ensure an optimum therapy. The control optimization of the medical devices occurs in the form of controller pre-settings and set value recommendations.

Until now, a controller setting is made exclusively by technical professionals in the context of maintenance works. This can be achieved, according to the invention, by self-learning systems and/or central data sets with an optimized selection of evidence-based parameter sets. The parameterization of control structures for different types of patients and types of surgeries is made by validated in silico models or process models available as HIL structures that are further improved by sufficient process data. HIL means hardware-in-line, i.e., technical representations of the operational environment by active elements with a controller acting in the background copy as identically as possible the surgery situation and reactions to device actions. This enables controlled and documented environmental conditions, in relation thereto the device control can reproducibly be optimized.

So far, technical process parameters were rarely analyzed and evaluated in the context of the clinical result (clinical outcome). According to the invention, an information collection, evaluation, and information-based process optimization is done in an anonymized manner and nevertheless tailored to the patient's individual needs. Therefrom follows that the automated linkage of pre-operative knowledge with the intra-surgical intervention carries the risk of individual overfittings. The determination of data and model structures as well as the non-linear optimization depend on the used data set and the chosen structure, with in particular the risk of overfitting. This is avoided, for example, according to prior art by a separation of training and validation data (leave-one-out-validation). Another promising solution according to prior art for preventing overfittings is offered by support vector machines (SVMs) from the statistical learning theory. Herein, individual data sets are considered as a random sample from an unknown basic population. The object is to find a description that is not optimal for the data set, but for the basic population. SVMs, therefore, have a significantly better generalization behavior than previously employed approaches.

The adaptation of the control to the patient requires, for reasons of safety and economy, the use of identification methods in closed loops. The sufficient process excitation for identification and the damping of the process excitation by the control are designed according to prior art.

The standard computer systems for use in the hospital environment are currently characterized by a strict separation of the real and virtual worlds. Future MIS system solutions will comprise intelligent surgery units with networked system components, intelligent sensors, and actuators. The embedded systems being widely used today do not meet these requirements. Future MIS system solutions will intensify the networking and intelligent control of different physical and virtual single systems. This will only be successful when the paradigm change of centrally controlled processes to holistic approaches is carried out with the inclusion of all available and usable information sources.

Solutions according to the invention are based on cyber-physical systems. Different from embedded systems, cyber-physical systems consist of networked system components autonomously coordinating themselves. These components consolidate to an automatically interacting, intelligent system environment and fuse integratively with reality. By definition, they are complex and consist of many heterogeneous and partly autonomously operating components and modules. Therefore, there is the necessity to create uniform definitions, standards as well as intelligent interfaces for the MIS, in order to safely and successfully operate medical device systems for the MIS through integrated management technologies.

There is also provided according to the invention a reliable and time-efficient method for identification of the medical devices used for the surgery. For example, in the field of arthroscopy, the flow properties of the employed dual roller pump, of the hose unit and the trocar-optics combination are to be determined, thereby an increase of the control performance in the intra-operative phase by consideration of a priori knowledge occurs, for example, the use of known flow properties of the identified medical devices. Currently, the recognition of used devices mostly takes place manually by the medical personnel. According to prior art, manufacturers identify their own accessories by suitable devices, such as RFID. The automated device recognition of all usable medical devices requires reliable sensor information and robust identification algorithms. A recognition is often, due to safety aspects, only possible in the pre-operative phase. Faulty recognition or a missing convergence of the identification algorithms may lead to erroneous assumptions for controlling the devices. A parameter estimation for the purpose of system identification occurs, according to the invention, in the closed loop. Furthermore, according to prior art, there are possibilities of indirect identification and direct identification.

Up to now, an adaptation of device-internal controls to the individual patient is made exclusively subjectively and manually by the medical professionals. Set value inputs are made manually by the medical personnel using individual experiences. For the derivation of recommendations for action, it is absolutely necessary to identify a correlation between relevant clinical process parameters and a clinical result and to derive a causality from the identified correlations. When this is not successful, there is the risk to derive erroneous recommendations for action. The determined causalities are provided, according to the invention, for the use in device adaptation (among others, for set value inputs, recommendations of accessories, selection of control methods, recommendations for action) in the medical device.

According to the invention, there is provided an assistance system that is available for the surgeon during every intervention in the field of minimally invasive surgery. This concept offers a high potential for further areas in the clinical environment and is to be used for the fields such as anesthesia, rehabilitation therapy or dialysis. As an extension of the assistance system, a feedback of information is imaginable, i.e., for example, the pressure in the joint is displayed as set value/guide information for the anesthesia doctors for blood pressure adjustment on the vital data monitor.

The networked medical device may also be used for a less time-critical surgery documentation. Background is that experiences about performed surgeries are not sufficiently evaluated, documented and published. This is also due to missing data that the networked medical device can output to a storage unit for later evaluation.

The device according to the invention consists, in one embodiment, of a medical device that can exchange, via an interface to the network of the hospital, for example, a safe network connection with medical devices in the surgery area, measurement data of sensors, information about the patient who is treated with the medical device, and operating parameters of the medical devices. This medical device includes a storage unit, wherein the data respectively assigned to a treatment case are collected and recorded in a structured manner. In case that operating parameters or patient data are only partially transmitted, e.g., due to connection problems, a data set is kept in the device that in such cases can be used as a fall-back level and enables a safe operation of the medical device, which has sent the data. With missing or interrupted connection, the medical device, too, will take recourse to this fall-back level. The medical device contains a computing unit that, due to superordinate rules and/or the evaluation of data entered via the interface, computes operating parameters and uses them for control/regulation. One kind of such a calculation may be a simple correlation of the incoming data sets existing in the medical device. Other linkages and calculations according to prior art that provide operating parameters for the further operation of the medical device, are also provided according to the invention. Alternatively, data sent to an external database or expert system, i.e. accessible via the interface, and operating parameters can be transmitted back.

A specific embodiment for such a medical product is a fluid pump that supplies a pressure in an artificial or created body cavity for expansion. This may be a liquid-filled body cavity, as is the case, for example, in urology, hysteroscopy, arthroscopy, as well as in procedures, in which tissue is opened, in order to enable access to certain regions and introduce implantations, as, for example, in the total extraperitoneal hernioplasty. According to the invention, in a liquid-filled body cavity, the intracavitary pressure can quickly be reduced by the use of suction instruments or therapy devices that remove tissue, thereby the body cavity becoming smaller. The communication of the sucking therapy devices to activate them via the already mentioned interfaces can enable the liquid pump to respond quickly, since otherwise the pressure drop will only be assigned with a delay of a suction situation. Further, according to the invention, it is provided to transmit or query the intensity of the suction by the identification of the employed device and the known parameters thereof or by the intensity of the device activity via the interface to the pump.

In another embodiment, the object of the fluid pump is to keep the field of vision free, e.g., from bleedings by open blood vessels and capillaries. This can be achieved by a tamponade effect, i.e., by a generated fluid counterpressure to the local blood pressure in the open vessels. At the same time, it is to be prevented to press the expansion fluid by overpressure into the blood vessels. According to the invention, it is, therefore, provided to detect the blood pressure with a measuring device, which is done, for example, by the anesthetist by means of a vital monitor that contains such a measuring device, and to transmit this information—but also others—via the interface to the medical device. When, thus, the fluid pump obtains the measured blood pressure of the patient, the local blood pressure at the body cavity is calculated, and the local blood pressure and/or the transmitted blood pressure are directly processed as control parameters for the pressure in the body cavity.

According to the invention, there is also provided the calculation of other vital parameters or patient data, for example, the inclusion of the patient's age to the maximum admissible pressure values of the fluid pump or the body-mass index calculated from height and weight that is taken into account with the weight of the abdominal wall for the expansion of the abdominal cavity (laparoscopy) as parameters for an increase of the set pressure.

The operating parameters of the medical devices processed according to the invention are in the case of a liquid pump the fluid pressure generated by the pump and/or the generated fluid flow, i.e., the supplied amount per time unit. According to the invention, there is further provided the control of the fluid temperature, which in the insufflator can occur by special heating hoses and in fluid pumps by accessories applying heat to the rinsing medium. These accessories can also be controlled by the temperature values determined by external sensors, such as, e.g., they are also connected to the vital monitor.

1. A medical engineering device for use in minimally invasive surgery (MIS), including
at least one interface to at least one other medical device, wherein the interface transmits measurement data and/or patient data,
at least one storage unit that stores the operating parameters obtained via the interface based on measurement data and/or patient data and that further permanently contains at least one data set of operating parameters as a fall-back level,
at least one computation unit connected via the interface in the other medical device, which based on the measurement data and/or the patient data computes the operating parameters of the medical device,
wherein the calculation of the operating parameters takes place under consideration of a priori data selected according to a predetermined criterion,
wherein the regulation of the operating parameters of the medical device takes place using the calculated operating parameters.

2. The medical engineering device according to claim 1, wherein the medical engineering device is a fluid pump.

3. The medical engineering device according to claim 2, wherein the fluid pump is an insufflator or a liquid pump.

4. The medical engineering device according to claim 1, wherein the at least one interface transmits measurement data of a blood pressure measurement device and/or other vital parameters of the patient and/or patient data of an electronic patient record.

5. The medical engineering device according to claim 1, wherein as operating parameters, fluid pressure, fluid flow, and/or fluid temperature are controlled.
